(19) United States
(12) Patent Application Publication
(10) Pub. No.: US 2008/0112974 A1
(43) Pub. Date: May 15, 2008

(54) METHOD FOR INDUCING MUCOSAL HUMORAL AND CELL-MEDIATED IMMUNE RESPONSES BY SUBLINGUAL ADMINISTRATION OF ANTIGENS

(75) Inventors: Cecil Czerkinsky, Nice (FR); Jan R. Holmgren, Vastra Frolunda (SE)

Correspondence Address: DARBY & DARBY P.C., P.O. BOX 770, Church Street Station, New York, NY, 10008-0770, US

(73) Assignee: Duotol AB, Vastra Frolunda (SE)

(21) Appl. No.: 11/851,998

(22) Filed: Sep. 7, 2007

Related U.S. Application Data
(60) Provisional application No. 60/843,125, filed on Sep. 8, 2006.

(51) Int. Cl. A61K 39/12; A61K 39/02; A61K 48/00

(52) U.S. Cl. 424/204.1; 424/234.1; 514/44

(57) ABSTRACT

Described are methods for inducing both a mucosal and a systemic immune response in the respiratory, digestive or urogenital tracts of a mammal to a microbial pathogen. The methods comprise topically administering onto the sublingual mucosa of the mammal an amount of an antigen effective to induce the mucosal and systemic immune responses and a pharmaceutically acceptable carrier or diluent. Pharmaceutical formulations and dosage forms for immunizing a mammal against a microbial pathogen to elicit a mucosal and systemic immune response in the respiratory, digestive or urogenital tracts are also described.

### PRIORITY

This application claims priority under 35 U.S.C. §119(e) to U.S. Provisional Application Ser. No. 60/843,125 filed Sep. 8, 2006, which is incorporated by reference herein in its entirety.

### FIELD OF THE INVENTION

The present invention pertains to methods for eliciting an immune response in mammals by sublingual administration of antigens and pharmaceutical formulations or dosage forms for use in the methods.

### BACKGROUND OF THE INVENTION

Conventional vaccines currently in use are administered parenterally and generally confer good protection against systemic disease through the induction of high titers of serum antibodies. Parenteral vaccines are suboptimal in that they fail to induce a local mucosal response that may prevent the early stages of an infection. Thus, the intranasal administration of a vaccine may provide a viable alternative to the parenteral route. Indeed, intranasal administration of non-replicating vaccine antigens, when formulated with an appropriate mucosal adjuvant (e.g., bacterial toxins), results in a vigorous local and systemic immune response. However, there is concern in terms of safety regarding this route of vaccine administration and in addition, it may require expensive delivery devices, e.g. nebullizers. Alternative needle-free vaccine delivery systems could aid in implementing mass immunization by increasing ease and speed of delivery, and by offering improved safety and compliance, decreasing costs, and reducing pain associated with vaccinations.

Mucosal surfaces are the main portal of entry of pathogens such as viruses and bacteria as well as allergens. The integrity of these surfaces relies upon the existence of a sophisticated immune system integrating innate and adaptive effector mechanisms which operate within mucosa-associated lymphoid tissues (MALT). The MALT immune system has evolved (i) to defend the host against harmful microorganisms, (ii) to maintain commensal microorganisms and (iii) to prevent harmful immune reactions against food and airborne antigens (Holmgren, J., and Czerkinsky, C. 2005. Nat Med 11:S45-53). Thus, the mucosal immune system can promote mucosal and systemic immune responses against pathogens or systemic immune hyporesponsiveness to harmless antigens, also called oral tolerance.

The sublingual mucosa of mice, in contrast to human sublingual mucosa, is a keratinized mucosa. This discrepancy between mice and humans has hampered development of drug delivery models in rodents. However, keratinization of the sublingual mucosa in mice may result from physical stimuli and not from fundamental differences with humans. Indeed, glandular and lymphoid apparatus related to the sublingual mucosa show features similar in mice and humans.

Mucosal vaccination, in contrast to parenteral vaccination, is of particular interest since stimulation of the mucosal immune system elicits secretory IgA (sIgA) and, under special conditions, mucosal cytotoxic T lymphocytes (CTL) (Staats, H. F., Bradney, C. P., Gwinn, W. M., Jackson, S. S., Sempowski, G. D., Liao, H. X., Letvin, N. L., and Haynes, B. F., 2001. J Immunol 167:5386-5394). Thus mucosal vaccination may prevent or limit transmission of infectious diseases through mucosae. In addition, mucosal vaccination with pertinent adjuvants elicits systemic humoral and cellular immune responses. Moreover, mucosal vaccination is non-invasive and is amenable to mass vaccination especially in developing countries.

Although the mucosal immune system is highly compartmentalized, it has been shown that mucosal immunization may promote sIgA responses in mucosal sites distant from the immunization site (Kunkel, E. J., and Butcher, E. C., 2003, Nat Rev Immunol 3:822-829). Oral immunization induces strong sIgA responses in the small intestine, ascending colon and mammary glands whereas this route of vaccine delivery is relatively inefficient for inducing sIgA response in the cervicovaginal mucosa and in the upper aerodigestive mucosae. Conversely, nasal immunization induces sIgA responses in the airways, nasal secretions and saliva but not in the gut. Moreover, nasal immunization evokes remote antibody responses in the female reproductive tract making it a route of choice in developing vaccination strategies against respiratory and sexually transmitted infectious diseases (Johansson, E. L., Rask, C., Fredriksson, M., Eriksson, K., Czerkinsky, C., and Holmgren, J. 1998. Infect Immun 66:514-520; Johansson, E. L., Wassen, L., Holmgren, J., Jertborn, M., and Rudin, A., 2001, Infect Immun 69:7481-7486). The attractiveness of nasal vaccination has been strengthened by the fact that it requires comparatively lower amounts (usually 10-100 fold) of antigens than oral (ingestion) vaccination, it induces stronger systemic antibody responses, and secretory antibody responses are usually more pronounced especially in the respiratory tract. However, development of nasal vaccination strategies in humans has been hampered by undesirable side effects, including effects on the central nervous system through interaction of various antigens including live viruses and recombinant adjuvants with the olfactory epithelium (van Ginkel, F. W., Jackson, R. J., Yuki, Y., and McGhee, J. R, 2000, J Immunol 165:4778-4782; Fujihashi K, Koga T, van Ginkel F W, Hagiwara Y, McGhee J R., Vaccine. 2002, Jun. 7; 20(19-20):2431-8).

An important challenge of mucosal vaccination is to define appropriate routes of immunization to generate site directed mucosal immune responses and systemic immune responses. Nasal immunization is of particular interest for mucosal vaccination since it evokes immune responses in the upper airways and remote immune responses in the genital tract. However, development of nasal vaccination strategies in human has been hampered because of undesired side effects affecting the central nervous system through the nervous olfactory bulb.

What is needed in the art are improved methods for eliciting an immune response in a mammal which are effective to induce a protective and effective antibody and cell-medicated immune response. The data presented herein demonstrates that sublingual immunization offers a viable alternative to nasal and oral immunization in vaccination against respiratory diseases or sexually transmitted diseases.

### SUMMARY OF THE INVENTION

In an effort to overcome the drawbacks of conventional systemic, intranasal, and oral vaccination strategies, the present inventors have discovered that the sublingual mucosa, a tissue that has received considerable interest for delivery of drugs and allergens into the blood circulation, can also serve as a site for inducing mucosal and systemic immune responses. In this regard, evidence that the sublingual administration of a non-replicating antigen can induce mucosal and systemic immune responses has been provided. Sublingual, literally ‘under the tongue’, from Latin, refers to a pharmacological route of administration in which certain drugs and macromolecules are entered directly into the bloodstream via absorption under the tongue. Many pharmaceuticals are prepared for sublingual administration. These commonly include cardiovascular drugs, steroids, barbiturates, some enzymes and increasingly frequently, certain vitamins and minerals.

The principle behind sublingual administration is as follows. When a chemical comes in contact with the mucous membrane covering the ventral part of the tongue and extending beneath the tongue to terminate at the junction with the gingiva of the inner surface of the inferior maxillary (or mandible), it diffuses into the epithelium beneath the tongue. This region contains a high density of blood vessels and capillaries, and as a result the substance quickly diffuses through the epithelium and enters the blood stream.

In theory, sublingual routes of administration have certain advantages over simple oral (swallowing) or gastrointestinal (GI) administration. This route is often faster, and entering a macromolecule or a drug into one's body sublingually ensures that the substance will rapidly come in contact with the sublingual epithelium prior to entry into the bloodstream. Macromolecules or drugs otherwise orally administered must resist the hostile environment of the gastrointestinal tract. This may mean a much greater percentage of the original substance is degraded either by enzymes in the GI tract or the acids it contains. Additionally, after GI absorption, the drug is sent to the liver where the drug may be extensively metabolized; this is known as the first pass effect of drug metabolism. Due to the degradative qualities of the stomach and intestine, or the barrier provided by mucins produced by cells of the GI tract, many substances, cannot be administered orally via ingestion.

Almost any form of substance is appropriate for sublingual administration, so long as in that form the substance can readily diffuse or penetrate through the sublingual epithelium in the mouth. Chemicals prepared as powders, solutions, or even aerosol sprays may all make use of this method. However, a number of factors, such as pH, molecular weight, and lipid solubility of a substance may determine whether the route is practical or not. Based on these properties, it is entirely possible that a drug, which will readily become a solution with saliva, simply diffuses too slowly (or not at all) in the sublingual mucosa to be effective.

Disclosed herein is the discovery that the sublingual mucosa, a readily accessible tissue, can serve as an inductive site of mucosal immune responses in the digestive, respiratory and genital tracts, and thus does not necessarily require entry of antigen into the bloodstream. The present invention discloses that sublingual application of an antigen can induce the recruitment of specific cells capable of presenting the antigen to the local immune system draining the sublingual mucosa. Such recruitment can be augmented by the co-administration of an adjuvant, which in turn results in enhanced mucosal immune responses. Sublingual co-administration of a soluble prototype protein antigen with cholera toxin adjuvant, has been found to induce vigorous immune responses in the airway mucosa and in the female reproductive tract. These responses were comparable to those seen after nasal immunization with similar doses of antigen and adjuvant. Importantly, and akin to nasal immunization, sublingual immunization induced antigen-specific cytolytic T cell responses in the lungs and in the genital tract. Moreover, sublingual immunization induced vigorous systemic humoral and CTL responses at doses comparable to those required for nasal administration. Such systemic responses include cell-mediated immune responses that elicit production of interferon gamma by T-lymphocyte cells. Overall, sublingual immunization was found to be more effective than oral (intragastric) immunization for inducing systemic immune responses and mucosal immune responses in the respiratory and genital tract mucosae. While oral immunization is considered the most effective route for inducing a local mucosal immune response in the digestive tract (Holmgren, J., and Czerkinsky, C. 2005. Nat Med 11:S45-53), we found that sublingual administration of a lysate of Helicobacter pylori (H. pylori), a bacteria that can cause gastritis and duodenal and stomach ulcers, was at least as effective as oral ingestion of the same lysate, to protect mice against colonization by H pylori.

Sublingual vaccination is thus an attractive alternative strategy to nasal and oral immunization for inducing potentially protective responses against digestive, respiratory and genital infections.

In one aspect, the present invention provides a method for inducing a mucosal immune response in the digestive, respiratory or urogenital tracts to a microbial pathogen in a mammal comprising topically administering onto the sublingual mucosa an amount of an antigen effective to induce the mucosal immune response, wherein the immune response comprises recruitment of MHC II-expressing cells in the sublingual mucosa, and a pharmaceutically acceptable carrier or diluent.

In another aspect, the present invention provides a pharmaceutical formulation or dosage form for immunizing a mammal against a microbial pathogen by topical administration onto the sublingual mucosa comprising an amount of an antigen effective for eliciting both a mucosal and systemic immune response in the digestive, respiratory and/or urogenital tracts, an amount of a mucoadhesive or bioadhesive effective for prolonging the contact between the antigen and the sublingual mucosa, and a pharmaceutically acceptable carrier or diluent.

In another aspect, the present invention provides a pharmaceutical formulation or dosage form for immunizing a mammal against a microbial pathogen by topical administration onto the sublingual mucosa comprising an amount of an antigen and an adjuvant which in combination are effective for eliciting a mucosal and systemic immune response against a microbial pathogen in a digestive, respiratory and/or urogenital tract, an amount of a mucoadhesive or bioadhesive effective for prolonging the contact between the antigen and adjuvant and the sublingual mucosa, and a pharmaceutically acceptable carrier or diluent.

These and other aspects of the present invention will be apparent to those of ordinary skill in the art in light of the present description, claims and drawings.

### BRIEF DESCRIPTION OF THE DRAWINGS

FIGS. 1A, 1B, 1C, and 1D. Histological analyses of the normal sublingual mucosa (1A). 1B shows immunohistochemical staining of the sublingual mucosa; note the presence of numerous leukocytes (CD45) in the thin lamina propria and beneath the epithelium and scattered Major Histocompatibility Class II (MHC class II)-stained cells in the lamina propria and occasionally in the basal epithelium. 1C shows immunoperoxidase staining of a transverse section of sublingual mucosa disclosing increased numbers of CD11c dendritic-like cells (see arrows in 1C) mainly located in the basal layer of the sublingual epithelium 2 hrs after topical administration of CT adjuvant, compared to the far right panel of 1B (labeled “MHC II”) which is without CT adjuvant treatment. 1D shows the kinetics of MHC class II stained cells in the sublingual epithelium zero, two, and six hours after topical sublingual administration of CT adjuvant.

FIGS. 2A and 2B. Sublingual immunization evokes systemic antibody responses. (A) Plasma antibody responses. Mice were immunized on days 0, 7, and 21 with OVA+/−CT. OVA-specific and CT-specific plasma Ig subclass titer were measured using an ELISA one week after the last immunization. Data are expressed as geometric mean antibody titer+SD, 5 mice per group. (B) Antibody-secreting cells in the spleen and in the draining lymph nodes. Spleen and draining lymph nodes were harvested one week after the last immunization. OVA-specific and CT-specific antibody-secreting cells were detected using an ELISPOT assay. Histogram bars represent the mean of specific antibody secreting cells (ASC) per million cells. Standard deviations were calculated from quadriplicate determinations from each experimental group of 5 mice per group.

FIGS. 3A, 3B, 3C, and 3D. Sublingual immunization elicits mucosal antibody responses.

Mice were immunized on days 0, 7, and 21 with OVA+/−CT. Mucosal fluids were collected one week after the last immunization. OVA-specific antibodies and CT-specific antibodies were measured in saliva (A), nasal washes (B), broncho-alveolar lavages (BAL) (C) and vaginal washes (D), using an ELISA. Data are expressed as the geometric mean Log 10 titer±SD, on groups of 5 mice.

FIGS. 4A, 4B, 4C, and 4D. Sublingual immunization elicits antibody-secreting cells in the lungs (A, B) and in the genital mucosa (C, D). Lungs and genital mucosa were harvested one week after the last immunization and single cell suspensions were prepared by enzymatic dispersion of sliced tissues. OVA-specific (A, C) and CT-specific (B, D) ASC were enumerated by an ELISPOT assay. Data are expressed as arithmetic mean number of specific ASC per 10⁶ cells±S.D. (standard deviation), on group of 4 mice. The results are representative of three independent experiments.

FIG. 5. Sublingual priming with CT and OVA induces CD4+ T cell proliferation in the SMLN. CFSE labeled DO11.10 transgenic CD4+ T cells were transferred on day 0 to Balb/c mice. Recipient mice were immunized sublingually or nasally with OVA+/−CT on day 1. Then, the submandibular lymph nodes were harvested at day 3, day 5, and day 7. Cellular proliferation was assessed by flow cytometry analyses of CFSE stained transgenic T cells.

FIGS. 6A and 6B. Sublingual immunization promotes T cell proliferative responses to OVA. BALB/c mice were immunized on day 0, 7, and 21 with OVA alone or OVA plus CT. Proliferating responses of submandibular lymph nodes cells or spleen cells stimulated with OVA in vitro were assessed one week after the last immunization. Data are expressed as arithmetic mean levels of incorporated radioactive thymidine (counts per minute, c.p.m.) ±S.D., determined on triplicate cultures of pooled cells from each experimental group consisting of 5 mice per group. Results are representative of three independent experiments.

FIG. 7. Sublingual immunization with CT and OVA induces mucosal and systemic cytolytic T cell responses. C57BL/6 mice were immunized at day 0, day 7, and day 21 with OVA+/−CT. On day 28, similar numbers of CFSEhigh, SYNFEKL peptide-pulsed and CFSElow, non pulsed splenocytes from naïve C57BL/6 were co injected i.v. into C57BL/6 mice. In vivo cytolysis was assessed in the spleen, the sublingual mucosa lymph node (SMLN) and the lungs. Histograms are gated on CFSE+ cells in recipient mice. Data represent the mean percentages of CFSElow or CFSEhigh cells among CFSE recovered donor cells, and were determined on 4 mice per group. This experiment is representative of three independent experiments.

FIG. 8 Sublingual immunization with H. pylori lysate antigen given either therapeutically to infected mice or prophylactically to uninfected mice which were subsequently infected can protect against H. pylori infection in a mouse model. The experiment is described in the Examples section. “I”=infection controls; “I+Sl”=infection followed by two sublingual (s.l.) immunizations; “Sl+I”=two s.l. immunizations followed by infection. Each treatment group comprises data from 10-20 mice, and combines data from half of the animals analyzed one week before the other half with closely similar results at both times. T test analyses for significance: p value for I vs I+Sl=<0.0001; same for I vs Sl+I; I+Sl vs Sl+I=0.012.

FIG. 9. Similar or better effect of sublingual immunization as compared with intragastric/peroral (p.o.) “therapeutic” immunization against H. pylori infection. The experiment is described in the Examples section. “I”=infection controls; “I+Po” and “I+Sl”=infection followed by two p.o. or two s.l. immunizations. Each treatment group comprises data from 14-20 mice, and combines data from half of the animals analyzed one week before the other half with closely similar results at both times. T test analyses for significance: p value for I vs I+Sl=<0.006; I vs I+Po=0.009; I+Sl vs I+Po=0.10.

### DETAILED DESCRIPTION OF THE INVENTION

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “sublingual administration” is defined herein as administering to the mucous membrane covering the ventral part of the tongue and extending beneath the tongue to terminate at the junction with the gingiva overlaying the inner surface of the inferior maxillary.

The terms “mucosal” or “mucosa” refers to mucous membranes covering the aerodigestive tract, urogenital tract, and respiratory tract as well as the eye conjunctiva, the inner ear and the ducts of all exocrine glands.

The phrase “induces a systemic immune response” refers to eliciting both an antibody-mediated response and a cell-mediated response. The antibody-mediated response involves secretory antibody production. The cell mediated response involves production of interferon-gamma and the development of a CTL response.

The phrase “induces a mucosal immune response” refers to eliciting both an antibody-mediated response and a cell-mediated response. The antibody-mediated response involves secretory antibody production. The secretory antibody production is characterized by IgA and/or IgG and/or IgM. The cell mediated response is a CTL response and/or a delayed-type hypersensitivity response.

It has now been discovered that immunization of the sublingual mucosa induces mucosal and systemic immune responses. The present invention provides a method for inducing both a mucosal and a systemic immune response in the digestive, respiratory or urogenital tracts to a microbial pathogen in a mammal comprising topically administering onto the sublingual mucosa an amount of an antigen effective to induce said mucosal and systemic immune response and a pharmaceutically acceptable carrier or diluent.

Although, the nasal route of immunization has been widely explored in terms of vaccination or tolerance, the sublingual route is poorly explored in terms of vaccination. These considerations have lead the present inventors to examine the effects of topically administering onto the sublingual mucosa an amount of an antigen or vaccine or an antigen and adjuvant on systemic and mucosal immune reactivities in a mammal. For example, antigen and cholera toxin (CT), the most potent mucosal immunogen and adjuvant, was administered sublingually to mice and the reactivities of the systemic and mucosal immune response measured. Additionally, in order to evaluate the potential of the sublingual mucosa in vaccination strategies, sublingual immunization was compared to extensively studied nasal immunization with ovalbumin (OVA) as a prototype antigen co-administered with the mucosal adjuvant cholera toxin.

The results presented herein indicate that the sublingual mucosa is an efficient inductive site of systemic humoral immune responses and systemic cellular immune responses in mammals. Interestingly, both sublingual immunization and nasal immunization with CT and OVA induced systemic immune responses to the same order of magnitude in terms of cellular proliferation, cytokine secretion, plasma antibodies and frequency of antibody secreting cells in the spleen. These results are consistent with a previous study that demonstrated induction of systemic immune responses after sublingual immunization with short peptides derived from Plasmodium falciparum antigens (BenMohamed, L., Belkaid, Y., Loing, E., Brahimi, K., Gras-Masse, H., and Druilhe, P., 2002, Eur J Immunol 32:2274-2281). These findings could be interpreted by the fact that small molecules such as short peptides can enter directly into the bloodstream and stimulate the systemic immune system following sublingual administration. However, the findings of BenMohamed et al. do not disclose or suggest that sublingual immunization induces a mucosal immune response in the digestive, respiratory or urogenital tract. Furthermore, BenMohamed et al. do not disclose or suggest that sublingual immunization induces a mucosal as well as a systemic effector CTL immune response.

Indeed, prior to the present invention, the potential of the sublingual mucosa to evoke mucosal immune responses had never been reported. As shown herein, sublingual as well as nasal administration of CT and OVA induced the recruitment of MHC-II expressing cells including dendritic cells in the sublingual mucosal epithelium and lamina propria. This recruitment was accompanied by a regionalized mucosal antibody response in the nasal tract, in saliva and in the lungs and vaginal secretions. The mucosal and systemic immune responses induced are specific to the digestive, respiratory and urogenital tracts. There were no responses detected in the intestine, indicating that the mucosal immune responses were not due to the uptake of antigens by the gastro-intestinal tract immune system. The mucosal and systemic immune responses induced may be measured in body fluids in a mammal treated with the claimed invention. Body fluids in which the mucosal and systemic immune responses may be measured include, but are not limited to, nasal tissues and/or fluids, blood, vaginal tissues and/or fluids, saliva, and lung tissues and/or broncho-alveolar lavage fluids.

Interestingly, sublingual as well as nasal immunizations were able to promote antibody responses in the lungs. Albeit antibody titers observed in broncho-alveolar lavages (BAL) may be due to transudation from the blood compartment, the presence of specific antibody-secreting cells in the lungs after sublingual immunization accounts for a truly localized antibody response. Moreover, sublingual immunization as well as nasal immunization with CT and OVA induced a CTL response in the lungs. Taken together, these results suggest that the nasal and the sublingual mucosae belong to a common upper respiratory immune compartment.

Unexpectedly, sublingual immunization with CT and OVA was able to induce a disseminated antibody response in the genital tract. Predominant secretory IgA and some IgG antibodies were detected in vaginal washes and substantial numbers of specific IgA-secreting cells were detected in the vaginal tract. These results suggest that the observed vaginal antibody responses reflect a dissemination of antibody secreting cells from the SMLN to the genital tract (Johansson, E. L., Rask, C., Fredriksson, M., Eriksson, K., Czerkinsky, C., and Holmgren, J., 1998, Infect Immun 66:514-520) or less likely, a dissemination of antigen-loaded dendritic cells to the genital tract (Belyakov, I. M., Hammond, S. A., Ahlers, J. D., Glenn, G. M., and Berzofsky, J. A., 2004, J Clin Invest., 113:998-1007). Previous studies demonstrated that a peculiar feature of nasal immunization is the ability to induce remote antibody responses in the genital tract. The mechanisms underlying the recruitment of effector cells to the genital tract after nasal immunization may involve specific homing (imprinting) of effector and memory cells during priming in the SMLN. Moreover, it has been previously demonstrated that the route of immunization could affect the homing phenotype of circulating plasmablast cells, hence controlling their recruitment to distant mucosal sites. Therefore, sublingual and nasal mucosae and/or SMLN may share cellular and molecular mechanisms affecting the homing phenotype of immune cells (Quiding-Jarbrink, M., Nordstrom, I., Granstrom, G., Kilander, A., Jertborn, M., Butcher, E. C., Lazarovits, A. I., Holmgren, J., and Czerkinsky, C., 1997, J Clin Invest 99:1281-1286; Kunkel, E. J., and Butcher, E. C. 2003, Nat Rev Immunol., 3:822-829).

In one embodiment, the present invention provides a method of inducing an immune response in a mammal comprising sublingually administering an amount of an antigen effective for eliciting said immune response against a microbial pathogen. Non-limiting examples of microbial pathogens include viruses, bacteria, mycoplasma, parasites, or fungi. Antigens which may be used in the claimed method include, but are not limited to, killed viruses, killed bacteria, live attenuated viruses, live-attenuated bacteria, protein antigens derived from viruses, bacteria, and parasites. Polysaccharides or conjugates comprised of polysaccharide and a protein can also be used. Non-limiting examples of microbial infections or pathogens against which the claimed method are useful include respiratory, buccal and genital pathogens, such as influenza virus, respiratory scyntitial virus, Hemophilusinfluenzae, Helicobacter pylori, Streptococcus pneumoniae, respiratory scyntitial virus, metapneumovirus, Streptococcus sobrinus (or also called Streptococcus mutans, causative agent of dental caries), periodontal pathogens, Neisseiria gonorrhea, Treponema pallidum (syphilis), Human Immunodeficiency Virus (HIV), Human Papilloma Virus, Chlamydia trachomatis, Candida sp and Mycoplasma pneumoniae.

In all embodiments, the antigen used in the claimed invention is immunogenic. In all embodiments, an antigen induces the recruitment of major histocompatibility (MHC) class II expressing antigen-presenting cells, including dendritic cells either by itself or co-administered with a compound capable of inducing recruitment of MHC class II expressing cells. Such compounds are referred to herein as “adjuvants”. Examples of such adjuvants inducing recruitment of MHC class II-expressing cells include, but are not limited to, cholera toxin and non-toxic mutant derivatives that have maintained adjuvant properties (See, e.g., Hagiwara Y, Kawamura Y I, Kataoka K, Rahima B, Jackson R J, Komase K, Dohi T, Boyaka P N, Takeda Y, Kiyono H, McGhee J R, Fujihashi K., J Immunol., 2006, Sep. 1; 177(5):3045-54; Yoshino N, Lu F X, Fujihashi K, Hagiwara Y, Kataoka K, Lu D, Hirst L, Honda M, van Ginkel F W, Takeda Y, Miller C J, Kiyono H, McGhee J R., J. Immunol., 2004 Dec. 1; 173(11):6850-7; Lomada D, Gambhira R, Nehete P N, Guhad F A, Chopra A K, Peterson J W, Sastry K J., Vaccine: 2004 Dec. 9; 23(4):555-65; Lycke N., Ann N Y Acad Sci., 2004, December; 1029:193-208; Boyaka P N, Ohmura M, Fujihashi K, Koga T, Yamamoto M, Kweon M N, Takeda Y, Jackson R J, Kiyono H, Yuki Y, McGhee J R., J Immunol., 2003 Jan. 1; 170(1):454-62; Yamamoto M, Kiyono H, Yamamoto S, Batanero E, Kweon M N, Otake S, Azuma M, Takeda Y, McGhee J R., J. Immunol., 1999, Jun. 15; 162(12):7015-21), Escherichia coli heat-labile enterotoxins (LT-I and LTII) (Kende M, Del Giudice G, Rivera N, Hewetson J. Vaccine., 2006, Mar. 15; 24(12):2213-21. Epub 2005 Nov. 15; Takahashi H, Sasaki K, Takahashi M, Shigemori N, Honda S, Arimitsu H, Ochi S, Ohara N, Tsuji T. Vaccine, 2006 Apr. 24; 24(17):3591-8; Ryan E J, McNeela E, Murphy G A, Stewart H, O'hagan D, Pizza M, Rappuoli R, Mills K H., Infect Immun., 1999, December; 67(12):6270-80; Cheng E, Cardenas-Freytag L, Clements J D., Vaccine, 1999, Aug. 20; 18(1-2):38-49), and their non-toxic mutant derivatives with adjuvant properties, certain oligodeoxynucleotides with known adjuvant activity (McCluskie M J, Davis H L., Vaccine, 1999 September, 18(3-4):231-7; Moldovean Z, Love-Homan L, Huang W Q, Krieg A M., Vaccine, 1998 July, 16(11-12):1216-24), pertussis toxin (Del Giudice G, Rappuoli R., Vaccine, 1999 Oct. 1, 17 Suppl 2:S44-52; Wilson A D, Robinson A, Irons L, Stokes C R., Vaccine, 1993; 11(2): 113-8), shiga toxin (Ohmura M, Yamamoto M, Tomiyama-Miyaji C, Yuki Y, Takeda Y, Kiyono H., Infect Immun., 2005 July, 73(7):4088-97), flagellin from Salmonella sp. (Pino O, Martin M, Michalek S M., Infect Immun., 2005 October, 73(10):6763-70). Chemokines such as CCL-20, CXCL14, CCR1 ligands and CCR 5 ligands, are known to attract MHC class II expressing dendritic cells and can thus be used as adjuvants in the present invention (See, e.g., Berlier W, Cremel M, Hamzeh H, Levy R, Lucht F, Bourlet T, Pozzetto B, Delezay O., Hum Reprod., 2006 May, 21(5):1135-42, Epub 2006 Mar. 10; Woltman A M, de Fijter J W, van der Kooij S W, Jie K E, Massacrier C, Caux C, Daha M R, van Kooten C., Am J Transplant., 2005 September, 5(9):2114-25; Cremel M, Berlier W, Hamzeh H, Cognasse F, Lawrence P, Genin C, Bernengo J C, Lambert C, Dieu-Nosjean M C, Delezay O., J Leukoc Biol., 2005 July, 78(1):158-66. Epub 2005 Apr. 14; Shurin G V, Ferris R L, Tourkova I L, Perez L, Lokshin A, Balkir L, Collins B, Chatta G S, Shurin M R., J Immunol., 2005 May 1, 174(9):5490-8, Erratum in: J Immunol., 2006 Mar. 15; 176(6):38401; Ferris, Robert [corrected to Ferris, Robert L]; Stumbles P A, Strickland D H, Pimm C L, Proksch S F, Marsh A M, McWilliam A S, Bosco A, Tobagus I, Thomas J A, Napoli S, Proudfoot A E, Wells T N, Holt P G., J Immunol., 2001 Jul. 1, 167(1):228-34). Other known adjuvants include Toll-like receptor ligands and agonists which are also referred to as pathogen-associated molecular pattern (PAMP) molecules. These PAMPs stimulate cells of the mammalian host to produce substances capable of amplifying the immune response. Examples of PAMPs are flagellin of bacterial flagella (Honko A N, Mizel S B. Immunol Res. 2005; 33(1):83-101), peptidoglycan (Takada H, Uehara A. Curr Pharm Des. 2006; 12(32):4163-72) and lipoteichoic acid (Palaniyar N, Nadesalingam J, Reid K B. Immunobiology. 2002 September; 205(4-5):575-94) of Gram-positive bacteria, lipopolysaccharide (LPS, also called endotoxin) of Gram-negative bacteria (Miyake K. Trends Microbiol. 2004 April; 12(4):186-92), lipid A and derivatives such as monophosphoryl lipid A (McCluskie M J, Weeratna R D. Curr Drug Targets Infect Disord. 2001 November; 1(3):263-71), double-stranded RNA from viruses (Kawai T, Akira S. J Biochem 2007 February; 141(2):137-45), and bacterial DNA containing CpG motifs (Klimnan D M. Int Rev Immunol. 2006 May-August; 25(3-4):135-54.

Non-limiting examples of analogs or agonists of PAMPs which have known adjuvant properties include synthetic oligonucleotides (ODN) containing uumethylated CpG motifs, double stranded RNA (poly I:C), imiquimod and CpG-rich oligonucleotides (CpG-ODN) (reviewed in Dalpke A, Zimmermann S, Heeg K. CpG-oligonucleotides in vaccination: signaling and mechanisms of action. Immunobiology 2001; 204: 667-76).

In all embodiments the antigen and/or the co-administered adjuvant elicit mucosal and systemic immune responses in the respiratory and urogenital tracts. In some embodiments, an antigen used in the claimed invention elicits mucosal and systemic immune responses in the digestive, respiratory and/or genital tracts. An antigen may be, but is not limited to, a protein, a polysaccharide, a lipid, a nucleic acid molecule, or conjugates thereof. An example of such antigens is the M2 protein or a derivative comprising the M2e ectodomain of the M2 protein, the hemagglutinin or any of the nuclear and envelope proteins and derivatives thereof of influenza virus. Such an antigen may be derived from an analog or derivative of a microbial pathogen. In some embodiments, the antigen is a polysaccharide or lipid derived from a microbial pathogen. In some embodiments, an antigen is a live virus or a live bacteria with limited ability to replicate in the sublingual mucosa, so-called “live-attenuated virus” virus or “live-attenuated bacteria”. In other embodiments, an antigen is a non-replicating microbial pathogen such as a killed virus or a killed bacteria.

In other embodiments, commercially available vaccines may be used in the claimed methods for topical administration onto the sublingual mucosa in amounts effective to induce a mucosal immune response, a systemic immune response and a CTL immune response in the respiratory and urogenital tracts. In all embodiments, the vaccine used in the claimed invention is immunogenic. Non-limiting examples of commercially available vaccines that may be used in methods of the claimed invention include tetanus vaccine, influenza vaccine, diphtheria vaccine, pneumococcal polysaccharide vaccine, pneumococcal polysaccharide-protein conjugate vaccine, Hemophilus influenzae polysaccharide-protein conjugate vaccine, meningococcal protein-polysaccharide conjugate vaccine, papilloma virus vaccine, acellular pertussis vaccine, papilloma virus vaccine. Non-limiting examples of commercially available live-attenuated vaccines that may be used for sublingual administration in methods of the claimed invention include measles virus vaccine, cold-adapted live-attenuated influenza virus vaccine, live-attenuated polio vaccine, Bacillus Calmette Guerin (BCG) vaccine. Examples of commercially available vaccines are listed in the following Table 1:

TABLE 1
Commercially Available Vaccines

| | | | Commercial |
|---|---|---|---|
| Disease | composition | company | name |
| Pertussis | purified pertussis | GSK | Infanrix ® |
| | antigens | | (combined |
| | | | vaccine) |
| | | Sanofi Pasteur | Pentavac ®, |
| | | | Pentacel ® |
| | | | (combined |
| | | | vaccine) |
| | Inactivated | Sanofi Pasteur | D.T.Coq. ® |
| | bacteria | | (combined |
| | | | vaccine) |
| Influenza | Inactivated virus | GSK | Fluarix ® |
| | | | vaccine |
| | Attenuated | MedImmune, | Flumist ® |
| | live virus | Inc | vaccine |
| Measles | Attenuated | Merck | Attenuvax ®, |
| | live virus | | Proquad ® |
| | | | (combined |
| | | | vaccine) |
| Mumps | Attenuated | Merck | Mumpsvax ®, |
| | live virus | | Proquad ® |
| | | | (combined |
| | | | vaccine) |
| Rubella | Attenuated | Merck | Meruvax ® II, |
| | live virus | | Proquad ® |
| | | | (combined |
| | | | vaccine) |
| Varicella | Attenuated | Merck | Varivax ®, |
| | live virus | | Proquad ® |
| | | | (combined |
| | | | vaccine) |
| Pneumococcal | Capsular | Merck | Pneumovax ® 23 |
| disease | polysaccharides | | vaccine |
| Tuberculosis | Attenuated live | Sanofi Pasteur | Monovax ® |
| | bacteria (BCG) | | vaccine |
| | BCG | Organon USA, | TICE ® BCG |
| | | Inc. | vaccine |
| | BCG | Aventis Pasteur | Theracys ® |
| | | Limited | vaccine |
| Tetanus | tetanus toxoid | Aventis Pasteur | Tetanus Toxoid |
| | | Inc. | USP vaccine |
| Diphtheria | diphtheria toxoid | Cantacuzino | Vaccin Difteric |
| | | Institute | Adsorbit |
| | | | vaccine |
| Papillomavirus | Purified virus-like | Merck | Gardasil ® |
| disease | particles of the | | vaccine |
| Cervical | major capsid | | |
| cancer | protein of HPV | | |
| prevention | | | |
| Polio | live attenuated | Lederle | Orimune ® |
| | polio virus | Laboratories | vaccine |
| Pneumococcal | pneumococcal | Wyeth | Prevnar ® |
| disease | polysaccharide- | Pharmaceuticals, | vaccine |
| | protein conjugate | Inc. | |
| | vaccine | | |

An amount of an antigen effective to induce recruitment of MHC class II-expressing cells in the sublingual mucosa and to induce a mucosal immune response, a systemic immune response and/or a CTL immune response may be readily determined by a skilled worker. The actual local concentration of the antigen needed for inducing recruitment of APCs capable of uptaking the antigen or vaccine and then migrating to the draining lymph nodes and tissues to stimulate T and B lymphocytes, which are responsible for either local (mucosal) or the systemic immune system, or both, is dependent on the nature of the antigen and can be determined by dose-response experiments. In fact, the instant application provides data showing that sublingual administration of an antigen together with a pertinent adjuvant induces recruitment of MHC II APCs (dendritic cells) in the sublingual mucosa and draining lymph nodes. In other instances, the antigen may not be capable per se of inducing the recruitment of MHC class II-expressing cells in the sublingual mucosa and may need to be co-administered with an adjuvant or a compound known to induce recruitment of MHC class II-expressing cells. The optimal amount of antigen to be administered may be determined by performing dose-response experiments well-known to those of ordinary skill in the art. For example, this can be done by using escalating doses of the antigen candidate, starting at very low doses then increasing until an immune response ensues and no side effects are observed.

The antigen may be used in effective amounts generally ranging between about 1.0 microgram and about 1000 micrograms. Higher concentrations are permitted subject to the amounts of physiological acceptability, but are not necessary. In some embodiments, the range of antigen concentrations used is generally ranging between about 10 micrograms and about 1 milligram per dose for proteins, glycoproteins and polysaccharides, as well as polysaccharide-protein conjugates. In one embodiment the antigen may be used in concentrations ranging between about 10 micrograms and about 500 micrograms. In the case of live-attenuated viruses, the effective amount ranges between about 10² and about 10⁸ TCID (tissue culture infectious dose), depending on the virus and its degree of attenuation. For attenuated bacteria, the effective amount ranges between 10⁴ organisms and about 10⁹ organisms, depending on the bacteria and its degree of attenuation. In the case of killed viruses and bacteria, the effective amounts are usually higher than for live organisms and the effective amounts range between 10⁵ and 10¹¹ viral particles and 10⁸ and 5×10¹¹ bacteria.

In another embodiment, the present invention provides a method of inducing mucosal and systemic immune responses in a mammal comprising topically administering to the sublingual mucosa an amount of an antigen or a vaccine, wherein said antigen or vaccine is effective for eliciting said immune response. In another embodiment, the present invention provides a method of inducing mucosal and systemic immune responses in a mammal comprising topically administering to the sublingual mucosa an amount of an antigen and an adjuvant which in combination are effective for eliciting said immune response. In some embodiments, the claimed invention may comprise one or more antigens and one or more adjuvants. In other embodiments, the present invention provides a method of inducing an immune response in a mammal comprising topically administering to the sublingual mucosa an amount of an antigen, wherein said antigen is effective for eliciting mucosal and systemic immune responses in the digestive, respiratory and urogenital tracts. In other embodiments, the present invention provides a method of inducing mucosal and systemic immune responses in a mammal comprising topically administering to the sublingual mucosa an amount of an antigen and an adjuvant which in combination are effective for eliciting an immune response in the digestive, respiratory and urogenital tracts.

The present invention also provides a pharmaceutical formulation or dosage form for immunizing a mammal against a microbial pathogen by topical administration onto the sublingual mucosa comprising an amount of an antigen effective for eliciting mucosal and systemic immune responses in the digestive, respiratory and/or urogenital tracts, an amount of a mucoadhesive or bioadhesive effective for prolonging the contact between the antigen and the sublingual mucosa, and a pharmaceutically acceptable carrier or diluent. In another embodiment, the present invention provides a pharmaceutical formulation or dosage form for immunizing a mammal against a microbial pathogen by topically administering onto the sublingual mucosa comprising admixing an amount of an antigen and an adjuvant which combination is effective for eliciting mucosal and systemic immune responses against a microbial pathogen in a digestive, respiratory and/or urogenital tract, an amount of a mucoadhesive or bioadhesive effective for prolonging the contact between the antigen and adjuvant and the sublingual mucosa, and a pharmaceutically acceptable carrier or diluent.

When formulated in a pharmaceutical composition, a therapeutic compound can be admixed with a pharmaceutically acceptable carrier or excipient. The phrase “pharmaceutically acceptable” refers to molecular entities and compositions that are “generally regarded as safe,” e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and, more particularly, in humans. The term “carrier” refers to a diluent, adjuvant, excipient, or vehicle with which the compound is administered. Such pharmaceutical carriers can further contain a nontoxic dose of a detergent. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers. Suitable pharmaceutical carriers are described in Remington: The Science and Practice of Pharmacy, Alfonso R. Gennaro, editor, 20^{th} ed. Lippingcott Williams and Wilkins: Philadelphia, Pa., 2000.

To enhance the presentation and/or availability of the antigen or antigen and adjuvant in combination for administration to the immune cells of the sublingual mucosa, mucoadhesive or bioadhesive agents may be used in the pharmaceutical formulations or dosage forms of the invention. Sublingual formulations containing mucoadhesives prolong the contact between the antigen or antigen and adjuvant and the sublingual epithelium. There are numerous mucoadhesives and bioadhesives that have been reported in the literature for sublingual delivery of therapeutic agents. In some embodiments, the antigen used in the claimed invention is administered with one or more mucoadhesive or bioadhesive agents. Non-limiting examples of mucoadhesives or bioadhesives that may be used in the antigen or antigen and adjuvant formulations or dosage forms of the invention include carbopols (CP934, and CP940), polycarbophil (PC), sodium carboxymethyl cellulose (SCMC) and pectin representing examples of the anionic type, chitosan (Ch) as an example of cationic polymer, and hydroxypropylmethyl cellulose (HPMC) as a non-ionic polymer, polyacrylic acid (PAA), capricol, polysaccharides, hyaluronic acid, chitosan, lectins; cellulose, methylcellulose, carboxymethylcellulose, hydroxypropyl methyl cellulose, sodium alginate, gelatin, pectin, acacia, and povidone. An effective amount of a mucoadhesive is an amount for prolonging the contact between the antigen or antigen and adjuvant and the sublingual mucosa. See, e.g., U.S. Pat. Nos. 5,908,637; 4,615,697; and 5,113,860.

Excipients with mucoadhesive properties for use with the antigen or antigen and adjuvant used in the claimed invention include, but are not limited to, pregelatinized starch, lactose, glycerol, propylene glycol, and various molecular weights of polyethylene glycol. Various biodegradable polymers that may be used in mucoadhesive or bioadhesive formulations include, but are not limited to, poly(lactides), poly(glycolides), poly(lactide-co-glycolides), polycaprolactones, polyalkyl cyanoacrylates, polyorthoesters, polyphosphoesters, polyanhydrides, and polyphosphazenes.

Use of adjuvants in combination with an antigen or a vaccine will enhance the magnitude of the immune response elicited. Adjuvants often selectively enhance humoral (antibody) and/or T cell-mediated responses, e.g. cytotoxic T cell responses. In some embodiments, an adjuvant is administered with the antigen or vaccine of the claimed invention. In some embodiments, an adjuvant is administered in combination with non-replicating antigens. Non-limiting examples of adjuvants for use in the present invention include cholera toxin (CT) and its non-toxic mutants, E. coli heat labile enterotoxin, pertussis toxin, and non-toxic mutants derived therefrom. CT, several non-toxic mutant derivatives of CT, B-subunits of CT (CTB) and E. coli heat labile enterotoxin, the A1 subunit of CT linked to an immunoglobulin fragment, and several non-toxic mutants thereof which selectively enhance mucosal immune responses when co-administered with an antigen by the nasal or the oral route. The inventors of the instant application are the first to show that CT and E. coli heat-labile enterotoxin and mutants therefrom function as adjuvants for enhancing both mucosal and systemic antibody responses as well as mucosal and systemic CTL responses after sublingual co-administration with an antigen.

The adjuvant may be used in an effective concentration generally ranging between about 0.01 mg/ml and about 1.0 mg/ml. The adjuvant administered with the antigen of the claimed method is co-administered in a volume ranging between about 0.01 ml and about 0.5 ml.

CT and CTB are commercially available from List Biological Laboratories, Inc., Campbell, Calif. 95008, USA. Recombinant (fCTB) SBL is commercially available from SBL Vaccin-Crucell, Stockholm, Sweden. E. coli LT and LTB may be obtained as described by Rask et al. APMIS 2000, 108: 178-86.

The present invention is set forth below in working examples which are intended to further describe the invention without limiting the scope thereof.

### EXAMPLES

### Materials and Methods

### Animals

Female BALB/c and C57BL/6 mice, 6 to 8 weeks old, were obtained from Charles River Laboratory (Les Oncins, France). Female DO11.10 transgenic mice, specific for OVA323_{—}339-peptide and 1-Ad-restricted TCR-αB, were treated subcutaneously with 10 mg of medroxyprogesteroneacetate (Depo-Provera) 5 days before vaginal samples collection.

### Immunizations

For sublingual immunization, mice were anesthetized with isoflurane and 5 μl of the solution was administered with a pipette under the tongue. The mice were then maintained 30 minutes without food and water. For nasal immunization, mice were anesthetized with isoflurane and 5 μl of solution was administered into each nostril.

### Sample Collection

Vaginal washes were collected on anesthetized mice one week after the last immunization by flushing twice with 50 μl of sterile PBS. Saliva was collected with a pipette on anesthetized mice after i.p. administration of pilocarpine (1 mg/mL) to promote saliva secretion. Nasal washes were collected after the mice were euthanized, by flushing retro in the nasal cavity with 200 μl of sterile PBS. Bronco-alveolar lavages (BAL) were collected after the mice were euthanized by flushing with 1 ml of sterile PBS through the trachea. All samples were clarified by centrifugation and stored at −20° C. Blood specimens were collected at the tail vein and were centrifuged at 3000 rpm at 4° C. for 15 min, from which plasma fractions were harvested and stored at −20° C.

### Titration of Specific Antibodies in Plasma and Mucosal Fluids

Plasma and mucosal antibodies against CT and OVA were determined by means of solid phase ELISA. Briefly, high binding 96-well polystyrene plates (Nunc) were coated with either CTB (2 μg/ml) or OVA (10 μg/ml) overnight at 4° C. Plates were blocked at room temperature (RT) for 90 min with 0.1 M pH 7.4 Phosphate buffer, 1% milk and 0.12% TritonX-100 (milk buffer). Serial dilutions of test samples in milk buffer containing 10 mM EDTA were incubated for 3 h at RT. After washing, the wells were incubated at RT for 1 h with affinity purified HRP-conjugated goat Abs to mouse IgG1, IgG2a, IgG2b and IgA (Southern Biotechnology Associates, Birmingham, Ala.). Solid phase-bound HRP activity was monitored spectrophotometrically after addition of tetramethylbenzidine (KPL, MD, USA). The titer of a sample was defined as the reverse of the highest dilution yielding an absorbance value at least equal to 3-fold that of background (no sample). The nonparametric Mann-Whitney's rank test was used to compare experimental groups for statistical differences.

### Cell Preparation

Prior to harvesting the lungs, mice were heavily anesthetized with pentobarbital and 125 IU of heparin (SIGMA) was injected intravenous. Blood was drawn directly from the heart and the mice were sacrificed by cervical dislocation. The tissues were perfused by injecting 15 ml of PB S/heparin (10 IU/ml) into the heart right ventricle until the lungs inflated and became clear. Then lungs, vagina, spleen and lymph nodes were dissected out and immersed in heparin-containing Dulbecco's phosphate buffered saline to allow ex vivo perfusion. Spleen and lymph node cells were isolated by pressing the organs through nylon net to obtain a single cell suspension. Erythrocytes were lysed with ammonium chloride. The lung cells were prepared by cutting the tissue into small pieces. Tissue fragments were digested in RPMI supplemented with collagenase A (0.5 mg/ml, Roche) DNase 1 (0.1 mg/ml, Roche), L-Glu and streptomycin-penicillin (100 μg/ml-100 U/mL) for 30 min at 37° C. The tissues were dissociated using a potter and the cell suspensions were passed through nylon net to remove tissue debris. Lung erythrocytes were lysed with ammonium chloride. The cervicovaginal cells were prepared as previously described. Briefly, the vaginal tissues were cut in small pieces. The pieces of tissue were digested in RPMI supplemented with 2 mg/ml collagenase-dispase (Roche,), 0.2 mg/ml DNase 1 (Roche,), L-Glu and penicillin-streptomycin (, 100 mg/ml-100 U/ml) for 45 min at 37° C. The cell suspensions were passed through nylon net to remove tissue debris. Vaginal erythrocytes were lysed with ammonium chloride.

### ELISPOT Assay

Cell suspensions from vagina, lungs, lymph nodes and spleen were analyzed for OVA- and CT-specific ASC by means of micromodified nitrocellulose-based ELISPOT assays (Czerkinsky, C. C., Nilsson, L. A., Nygren, H., Ouchterlony, O., and Tarkowski, A; 1983, J Immunol Methods 65:109-121). Briefly, nitrocellulose-bottom 96-well plates (Millipore) were coated overnight at 4° C. with OVA (30 μg/ml, Sigma) or with 1.5 μM GM1 ganglioside (Sigma) followed by 5 μg/ml CTB 2 h at 37° C. After washing with sterile PBS, antigen-coated wells were blocked with 10% FCS in DMEM for 1 h at 37° C. Immediately after isolation, MNC suspensions were added in quadruplicate to the antigen-coated wells and incubated for 5 h at 37° C. in a moist atmosphere with 5% CO₂. After extensive washing of wells with PBS, plates were soaked and washed thoroughly in PBS Tween 0.1%. HRP-conjugated goat Abs to mouse IgG and IgA (Southern Biotechnology Associates, Birmingham, Ala.) were incubated overnight at 4° C. in PBS supplemented with 5% FCS and 0.1% Tween. After washing with PBS 0.1% Tween® detergent, spots were developed by using 0.5 mg of 3-amino-9-ethylcarbazole (Sigma) per ml and 0.015% (vol/vol) H₂O₂ in 0.1 M sodium acetate (pH 5.0). Spots were enumerated using an a videoscan image analyzer (CTL, Cleveland, USA) after calibration for size and intensity to exclude artifacts.

### In Vivo T Cell Proliferation Assays

A total of 6×10⁶ T lymphocytes (>85% CD3+) isolated from the spleen and lymph nodes of DO11.10 mice were labeled with 4 μM Carboxy Fluoroscein Succinimidyl Ester (CFSE) vital dye/stain (Molecular Probes) and transferred i.v. in recipient syngeneic BALB/C mice. On day 1 following adoptive transfer, recipient mice were immunized with 200 μg of OVA with or without 2 μg of CT, or with PBS alone. Submandibular draining lymph nodes were harvested 2 days, 4 days or 6 days after immunization and cellular proliferation was ascertained by FACS analyses of CFSE profiles after gating live KJ126+ transgenic T cells.

### In Vitro Cellular Proliferation

Cellular OVA-specific proliferative responses were determined on triplicate cultures of lymph node (LN) or spleen cell suspensions. Cells were seeded at 4×10⁵ cells per flat-bottom well of 96-well culture plates (Falcon; BD Biosciences, Franklin Lakes, N.J.). In LN cell suspensions, naïve spleen cells (0.5×10⁵ cells) were added into the wells as a source of APC. After incubation at 37° C. with 5% for 72 h in the presence or absence of 2 mg/ml OVA (Sigma), cultures were pulsed for another 18-h period with 1 μCi [³H]thymidine per well. Cultures were harvested onto a filter (cell harvester,) and the extent of radioactive thymidine incorporated was measured with a beta scintillation counter (Wallac).

### In Vivo CTL Assay

In vivo CTL assay was performed essentially as described with minor modifications (Barber, D. L., Wherry, E. J., and Ahmed, R., 2003, J Immunol 171:27-31). Spleen cell suspensions were prepared from C57BL/6 mice. Spleen cell suspension was then washed and split into two populations. One population was labeled with 4 μM CFSE (CFSEhigh cells), then pulsed with 10⁻⁶M OVA257-264 SIINFEKL peptide (Proimmune) and incubated at 37° C. with 5% CO₂ for 45 min. The second control target population was labeled with 0.4 μM CFSE (CFSElow cells) then incubated at 37° C. with 5% CO₂ for 45 min without peptide. CFSEhigh cells and CFSElow cells were mixed in equal number, such that each mouse received a total of 20×10⁶ cells in 200 μl of PBS. Cells were injected intravenously into immunized mice 5 days after the last immunization. Mice were sacrificed 24 h or 48 h after transfer and spleen, SMLN and lungs were harvested. Cell suspensions were analyzed by flow cytometry, and pulsed and unpulsed populations were detected by differential CFSE staining profiles. Percentage of specific lysis was calculated according to the formula: (1−(ratio unprimed/ratio primed)×100), in which the ratio unprimed=percentage of CFSElow/percentage of CFSEhigh cells remaining in non-immunized recipients, and ratio primed=percentage of CFSElow/percentage of CFSEhigh cells remaining in immunized recipients.

### Immunohistochemistry

Sublingual mucosa was dissected, enrobed in 10% sucrose, then placed in Shandon Cryomatrix™ (ThermoElectron corp.) embedding medium and immediately snap-frozen by placing the tissue on a culture plate floating on liquid nitrogen. Cryostat sections (7 μm thick) were placed onto Superfrost™ Plus slides (Fisher Scientific, Santa Clara, Calif.), air dried for 24 hr at room temperature, then fixed in cold acetone for 5 min, and rehydrated in phosphate-buffered saline (PBS) for 10 min.

For enzyme-based immunohistochemistry, sublingual sections were incubated with streptavidin-biotin blocking kit (Vector Laboratories, Burlingame, Calif.) containing 10% normal goat serum (Jackson ImmunoResearch Laboratories, Inc., West Grove, Pa.) according to the manufacturer's protocol, then 1% H₂O₂ in sodium azide/PBS for 15 min to block endogenous peroxidase. Following a 10 min rinse with PBS, sections were incubated for 3 hr in a humidified chamber at room temperature with primary monoclonal antibodies (BD Pharmingen) including biotinylated anti-I-A/I-E mAb (2G9), purified anti-CD11c mAb (HL3), purified anti-CD45 (LCA) mAb (30-F11), purified anti-Gr-1 mAb (RB6-8C5).

Specific binding was revealed by 30 min incubation with secondary multiple absorbed biotinylated goat anti-hamster IgG (Jackson ImmunoResearch Laboratories) for CD11c staining or goat anti-rat IgG (BD Pharmingen) antibodies followed by 30 min incubation with Vectastain® ABC staining kit (Vector Laboratories). Colorimetric reaction was performed with AEC substrate for peroxydase kit (Vector Laboratories) and counterstained with hematoxylin (DakoCytomation), rinsed with distilled water, and mounted with Gel Mount® mounting medium (Biomeda corp. Foster city, Calif.).

Isotype-matched negative control antibodies were used to stain tissues to ensure the specificity of positive staining reactions.

### Experimental Infection with H. pylori

Infection of the stomach with H. pylori can cause gastritis and duodenal and stomach ulcers. H. pylori infection may also cause or is at least a strongly associated risk factor for stomach cancer. To prevent H. pylori infection or disease development from the infection, especially in the light of the rapidly increasing rate of antibiotic resistance among H. pylori clinical isolates, there is great interest in the potential of developing an effective H. pylori vaccine for either prophylactic or therapeutic use (prophylactic meaning that the vaccine is given to an individual before infection has occurred, and therapeutic meaning that the vaccine is given to an already infected individual).

We tested the potential of sublingual (s.l.) immunization, given prohylactically or therapeutically, against H. pylori infection in a mouse model. C57/B1 female mice, age 6-8 weeks at the start of the experiments, were either 1) first given two s.l. immunizations with H. pylori lysate antigen together with cholera toxin (CT) as adjuvant at two weeks interval and then two weeks later infected with 1×10⁹ H. pylori bacteria (the SS1 strain), or 2) first infected and then two weeks later given the two s.l. immunizations (0.5 mg lyophilized lysate protein and 5 micrograms CT were given each time and applied under the tongue in 3 aliquots of 5 microliter each at ca. 10 minutes intervals). Two or three weeks after the last intervention (immunization or infection) the mice were sacrificed and the number of H. pylori bacteria in the stomach determined. Concurrently infected but not immunized mice served as controls. The infection model, the preparation of the H. pylori lysate antigen used for immunization, the determination of the infection load (number of bacteria in the stomach) and other experimental details have been described in previous reports (Raghavan S, Hjulstrom M, Holmgren J and Svennerholm A-M, Infection and Immunity 70:6383-6388, 2002; Nyström J, Raghavan S and Svennerholm A-M, Microbes and Infection 8:442-449, 2006).

We also compared the effect of s.l. immunization with that of corresponding intragastric/peroral (p.o.) immunization, and tested this in the therapeutic model system described. The s.l. immunizations in H. pylori infected mice were identical to those described in the experiment above, and at the same times other infected mice were instead immunized with the same amounts of lysate antigen and CT in 300 microliter bicarbonate solution through a baby feeding catheter. The extent of infection was determined at 7 and 8 weeks after infection (3-4 weeks after the last immunization).

### Results

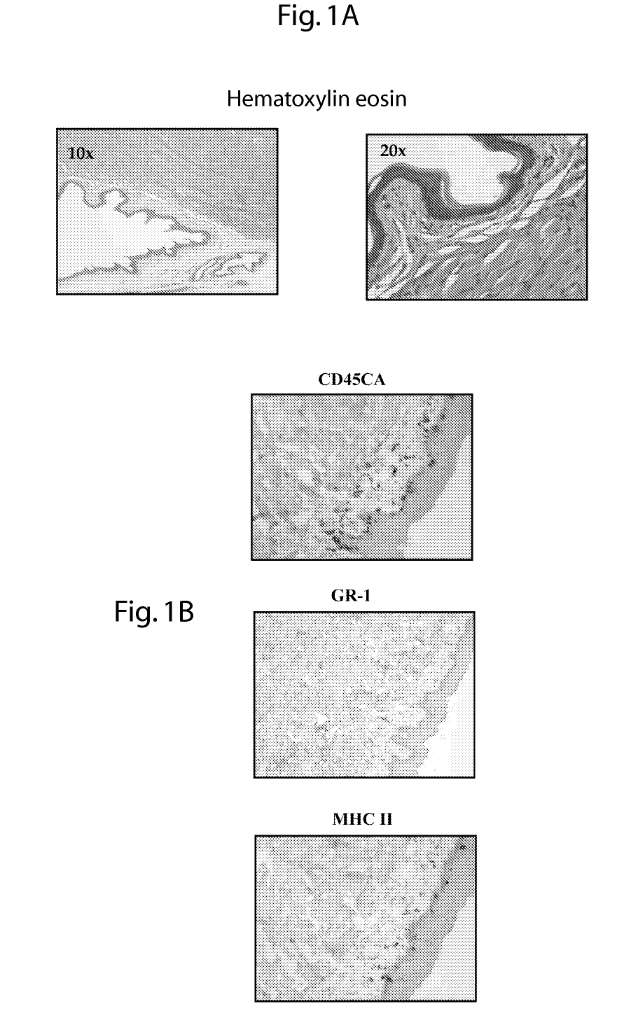

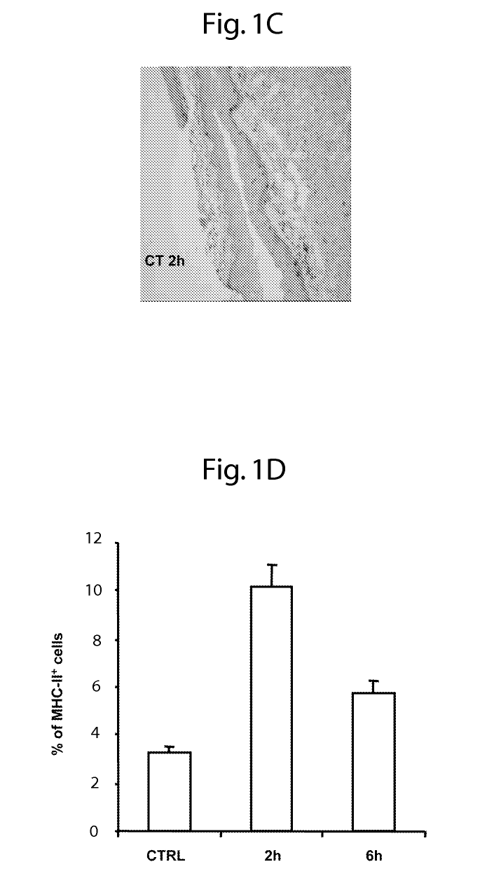

Hematoxylin-eosin staining of the sublingual mucosa was performed to characterize putative cellular alterations in the sublingual mucosa after sublingual administration of CT. CT neither altered the morphology of the mucosa nor induced an inflammatory infiltrate. In addition, we observed recruitment of MHC class II+ cells into the SLM within 2 h after sublingual administration of CT. Histological analyses of the sublingual mucosa in mice shows a keratinized epithelium overlying a sub mucosa disclosing few capillary vessels, leukocytes and fibroblast nuclei (FIG. 1A). Immunohistochemical analyses of the sublingual mucosa of untreated mice indicated the presence of numerous leucocytes in the lamina propria (FIG. 1B). More specifically, it is interesting to note the presence of MHC class II+ cells and GR-1+ cells in the lamina propria and in the epithelium of the sublingual mucosa (FIG. 1B). Typical appearance of a transverse section of sublingual mucosa obtained 2 hrs after topical administration of CT adjuvant and disclosing increased numbers of CD11c dendritic-like cells mainly located in the basal layer of the sublingual epithelium (immunoperoxidase staining (FIG. 1C). FIG. 1D shows the kinetics of MHC class II stained cells in the sublingual epithelium after topical sublingual administration of CT adjuvant.

### Sublingual Immunization Evokes Vigorous Systemic Antibody Responses.

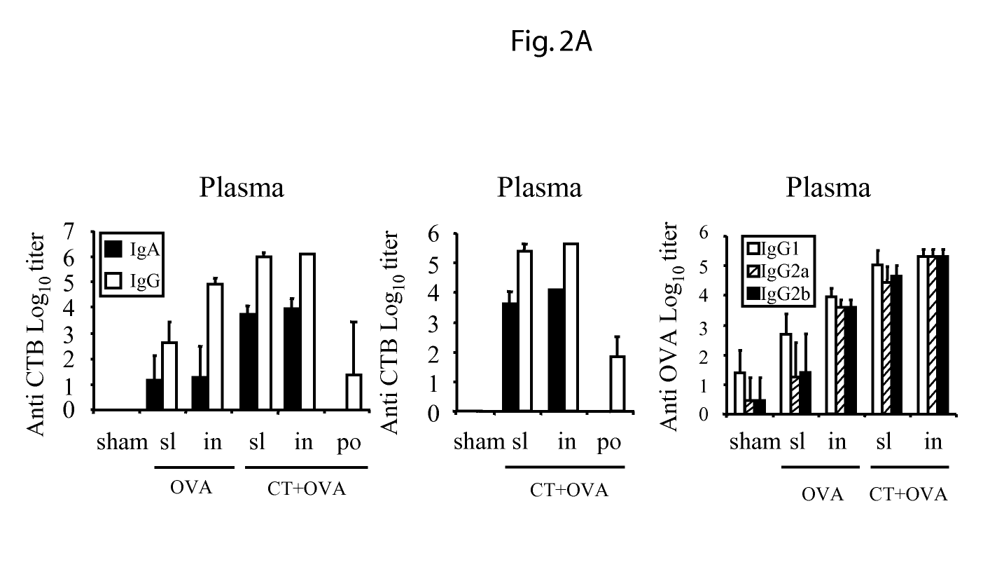

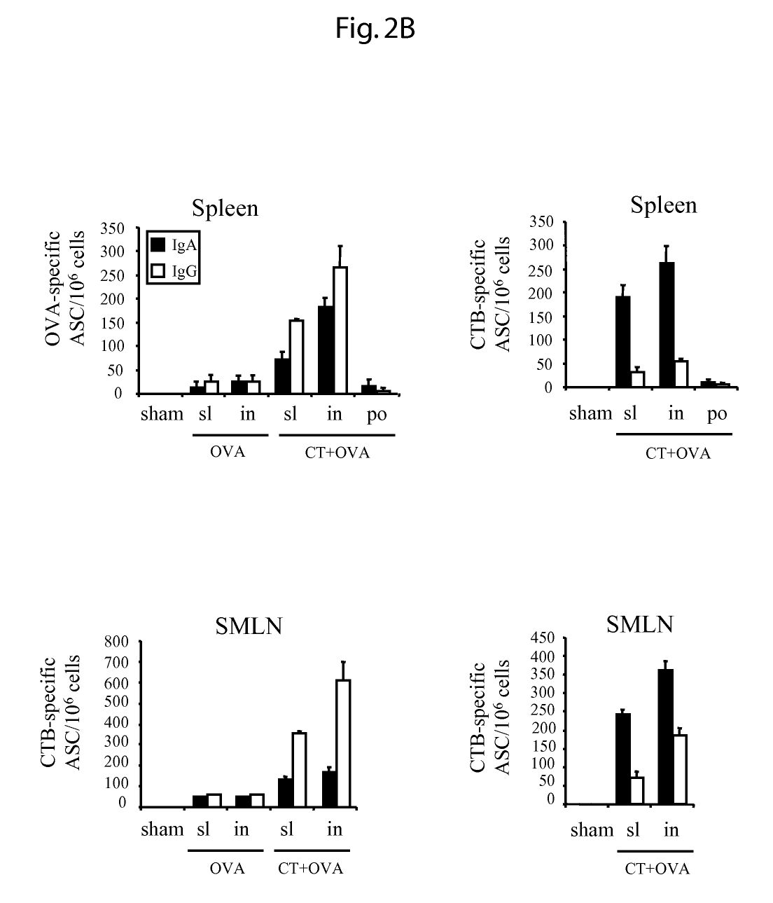

Sublingual administration of OVA admixed with CT adjuvant was as efficient as nasal immunization for inducing IgG and IgA antibody responses in serum (FIG. 2A). All mice responded to CT and OVA after sublingual and nasal immunization with CT and OVA. Moreover, sublingual and nasal immunization induced a mixed IgG subclass antibody response in the serum (FIG. 2A).

Since antibody responses measured in plasma after sublingual immunization could result from ingestion of the antigens, experiments were conducted to address this possibility. Gastric administration of a comparable amount and volume of CT given by intubation with PBS without antacid buffer failed to induce detectable antibody responses in the serum (FIG. 2A). Furthermore, ELISPOT analysis of cell suspensions from the small intestine and spleen of animals fed CT failed to detect any appreciable antibody-secreting cell (ASC) responses to CT. Immunofluorescence analysis of sections prepared from intestinal specimens collected 1 hr and 2 hrs after sublingual administration of FITC-conjugated CT failed to detect any staining in the small intestine. In contrast, intragastric administration of the conjugate showed staining located in isolated villi in the proximal segment of the duodenum (not shown). Taken together, these findings indicate that immune responses observed after sublingual administration of antigen under the conditions used herein did not result from the intestinal uptake of antigens. (FIG. 2B).

Sublingual as well as nasal immunization with CT and OVA induced CT-specific and OVA-specific ASCs in the spleen (FIG. 2B). Of note, the proportion of specific IgG- and IgA-ASCs seemed to depend on the antigen administered. Thus, CT-specific IgA ASCs were predominant compared to CT-specific IgG ASCs whereas OVA-specific IgG-ASCs were predominant compared to OVA-specific IgA ASCs.

### Sublingual Immunization Induces Regional Antibody Responses.

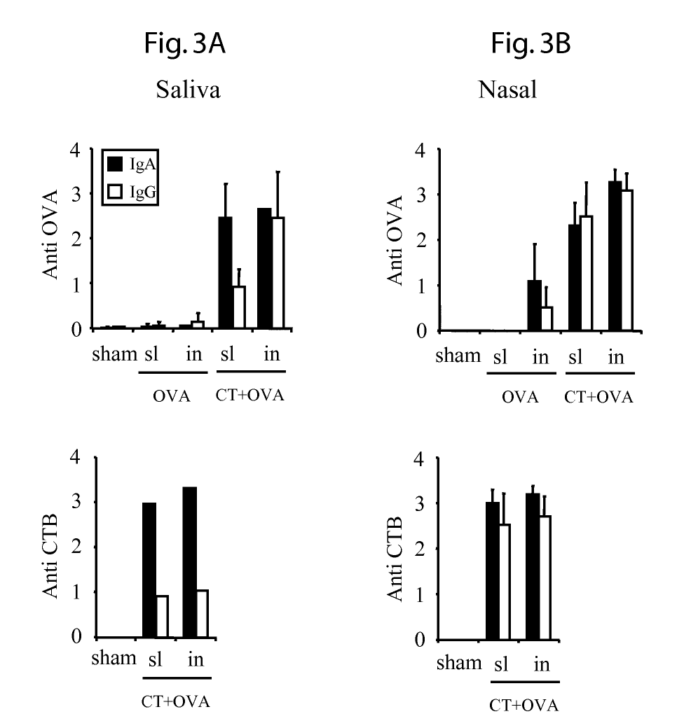

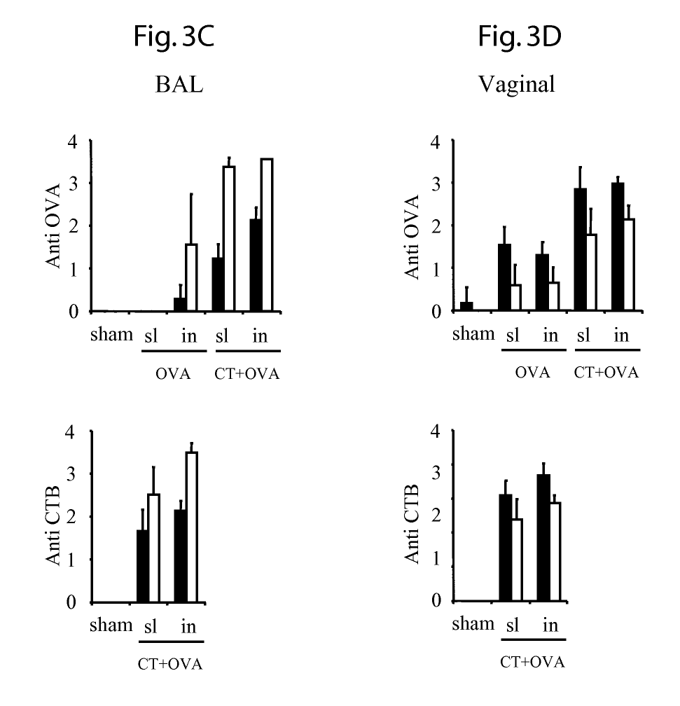

ELISA measurements of specific antibodies in nasal secretions, saliva, and BAL were performed at various times after co-administration of OVA and CT given by the sublingual or by the nasal route. Sublingual as well as nasal immunization induced anti-OVA and anti-CT antibody responses in all secretions examined (FIG. 3). Interestingly, sublingual immunization with CT plus OVA induced high levels of OVA-specific and CT-specific IgA and IgG antibodies in nasal secretions (FIG. 3B). Moreover, levels of mucosal antibodies in nasal secretions after sublingual immunization with CT and OVA were comparable to those seen after nasal immunization with CT and OVA.

Antibody response in the lungs after sublingual immunization was assessed in BAL by ELISA. Sublingual immunization with CT and OVA was as efficient as nasal immunization to induce specific IgA and IgG antibody responses in BAL (FIG. 3C); however, in BAL specific IgG responses were predominant compared to IgA.

A peculiar but characteristic feature of nasal immunization is to generate immune responses in the reproductive tract mucosa.

Antibody-secreting cells were measured by ELISPOT one week after the last immunization in cell suspensions from sub-maxillary (SLN), mesenteric lymph nodes (MLN) and iliac lymph nodes (ILN). After sublingual immunization with CT and OVA but not with OVA alone, the frequency of ASCs increased in draining SLN (FIG. 2B) being negligible in both MLN and iliac lymph nodes (data not shown). ASC numbers in SLN of animals immunized sublingually with CT and OVA were similar to those of animals given the same dose by the nasal route. Like spleen ASC responses, SLN ASC responses to OVA and to CT differed with regard to isotype distribution with a predominance of IgA-ASCs to OVA and of IgG-ASCs to CT (FIG. 2B).

Genital antibody response in the genital tract after sublingual immunization were evaluated. It has been shown previously that nasal immunization can elicit remote antibody responses in the genital tract by promoting the recruitment of antibody secreting cells in the genital mucosa (Johansson, E. L., Rask, C., Fredriksson, M., Eriksson, K., Czerkinsky, C., and Holmgren, J., 1998, Infect Immun 66:514-520). Sublingual immunization as well as nasal immunization elicited specific antibodies in the vaginal secretions that were mainly secretory IgA (FIG. 3D).

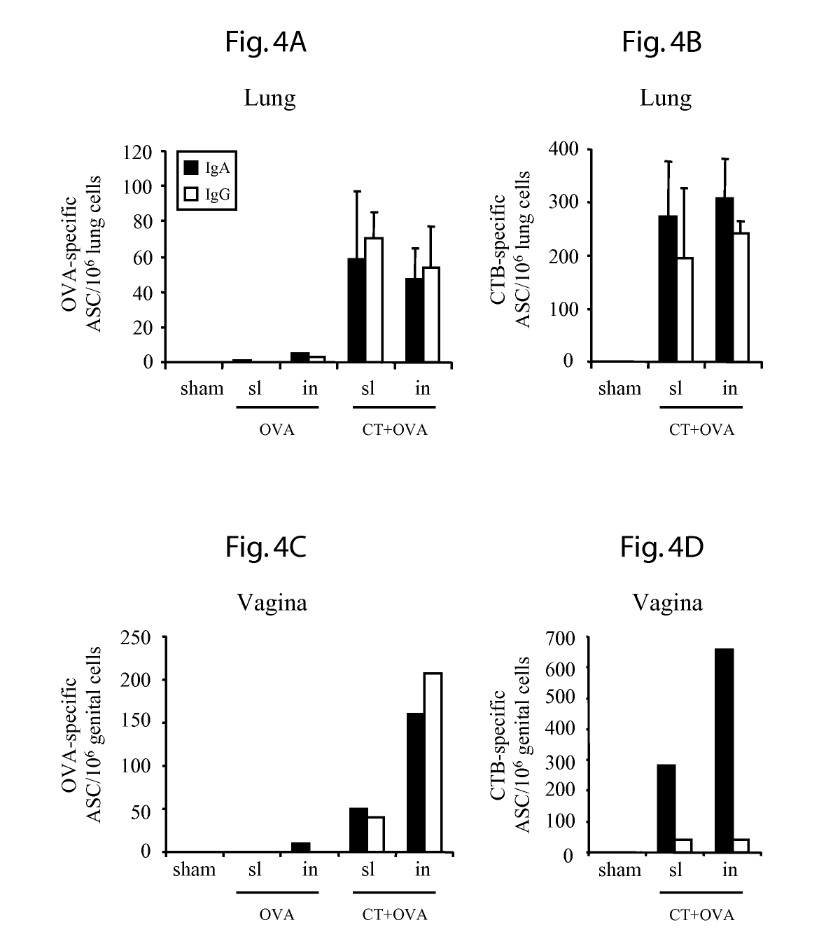

We evaluated by ELISPOT the contribution of local antibody-secreting cells in the production of mucosal antibodies in BAL and vaginal secretions. Sublingual immunization with CT and OVA induced predominantly specific IgA-secreting cells in the lungs (FIG. 4A). Frequencies of specific antibody secreting cells in the lungs after sublingual immunization were equivalent to frequencies of specific antibody-secreting cells after nasal immunization. Moreover, sublingual immunization promoted recruitment of specific antibody secreting cells in the genital mucosa (FIG. 4B) and few specific antibody-secreting cells were detected in the ILN (data not shown).

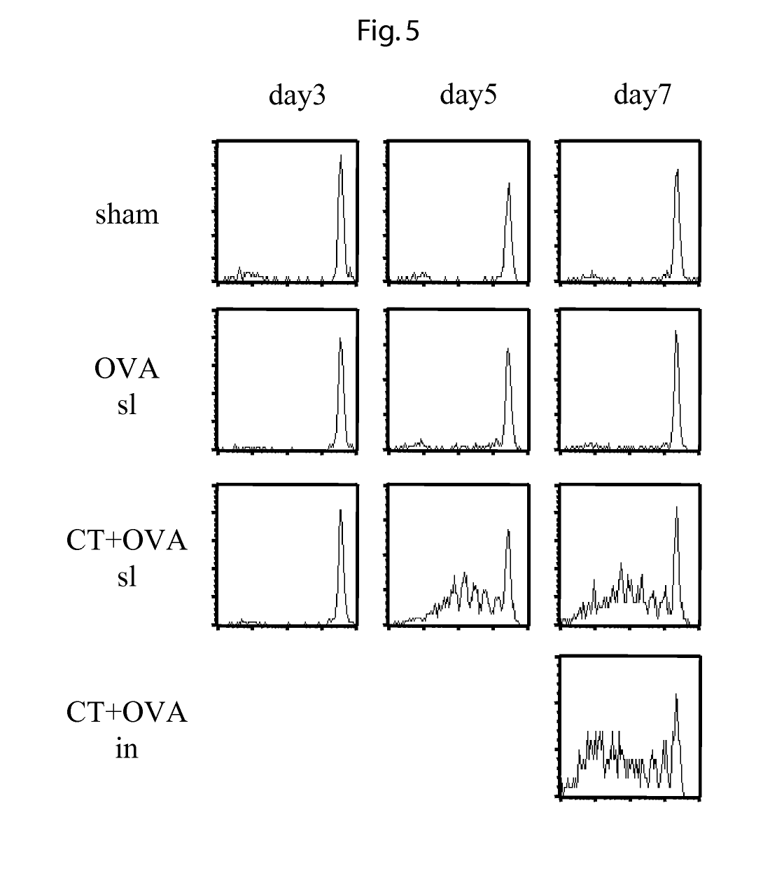

It has been shown that CT is not only a mucosal adjuvant of antibody responses but also a potent mucosal adjuvant of T cell-mediated cellular immune responses. To evaluate the potential of the sublingual mucosa to elicit cellular responses we assessed priming of CD4+ T cell responses in the SMLN after sublingual priming. A single sublingual immunization with CT and OVA in BALB/c mice who had previously been infused with DO11.10 CD4+ T cells promoted the proliferation of DO11.10 CD4+ T cells in the SMLN (FIG. 5). No detectable proliferation of DO11.10 CD4+ T cells was observed in the spleen (data not shown) suggesting that CD4+ T cells priming occurred mainly in the draining submandibular LN after sublingual priming.

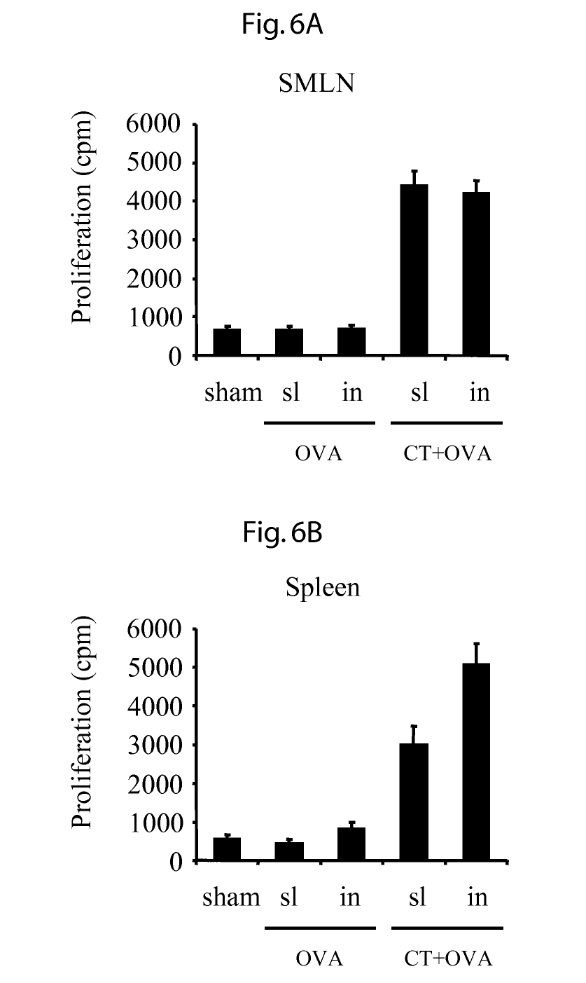

We also evaluated cellular responses after a sublingual prime boost regimen of immunization. One week after the last immunization, cellular proliferation from draining lymph nodes or spleen cell suspensions re-stimulated with OVA in vitro was measured. Sublingual immunizations with CT and OVA elicited proliferative responses of SMLN cell suspensions upon re-stimulation with OVA in vitro (FIG. 6A). In contrast to T cell priming experiments, repeated sublingual immunization with CT and OVA induced proliferative responses of spleen cell suspensions re-stimulated with OVA in vitro (FIG. 6B). Interestingly, sublingual and nasal immunization with CT and OVA induced similar proliferative responses in vitro.

Sublingual administration of CT induced both Th1 (T helper cell type 1) and Th2 (T helper cell type 2) responses. Th1 (IFN-γ) and Th2 (IL-4 and IL-10) cytokine secretions were measured one week after the last immunization in cell suspensions from draining lymph nodes or from spleen upon OVA re-stimulation in vitro. Interestingly, sublingual administration of CT induced production of IFN-γ, IL-4 and IL-10 by SMLN cells and spleen cells (TABLE 2).

TABLE 2
Cytokine Secretion: SLI with CT and OVA promotes Th1/Th2 cytokine
secretion in vitro under OVA stimulation^{a}

| | | | | Splenocytes | | |
|---|---|---|---|---|---|---|
| | SMLN cells | | | cytokine | | |
| sublingual | cytokine secretion^{b} | | | secretion^{b} | | |
| immunization | IFN-γ | IL-4 | IL-10 | IFN-γ | IL-4 | IL-10 |
| sham | ND^{c} | ND | ND | ND | ND | ND |
| OVA | ND | ND | 23.8 | ND | ND | 23.8 |
| OVA + CT | 6919.1 | 75.4 | 813.8 | 9413.8 | 38.7 | 466.8 |

^{a}Mice were immunized on day 0, 7 and 21. Cytokine secretion of submandibular and cervical lymph node cells (A) or spleen cells (B) stimulated with OVA in vitro were assessed one week after the last immunization.
^{b}Values are means determined in triplicate culture for each group of immunization, 4 mice per group.
^{c}N.D., not detectable (below detection limit of the assay)

Th2 responses are required for supporting secondary antibody responses, especially mucosal antibody responses. Thus, the ability of sublingual immunization to induce strong regional (SMLN) and systemic (spleen) Th2 responses is consistent with our finding that this method of immunization is efficient for inducing mucosal and systemic antibody responses.

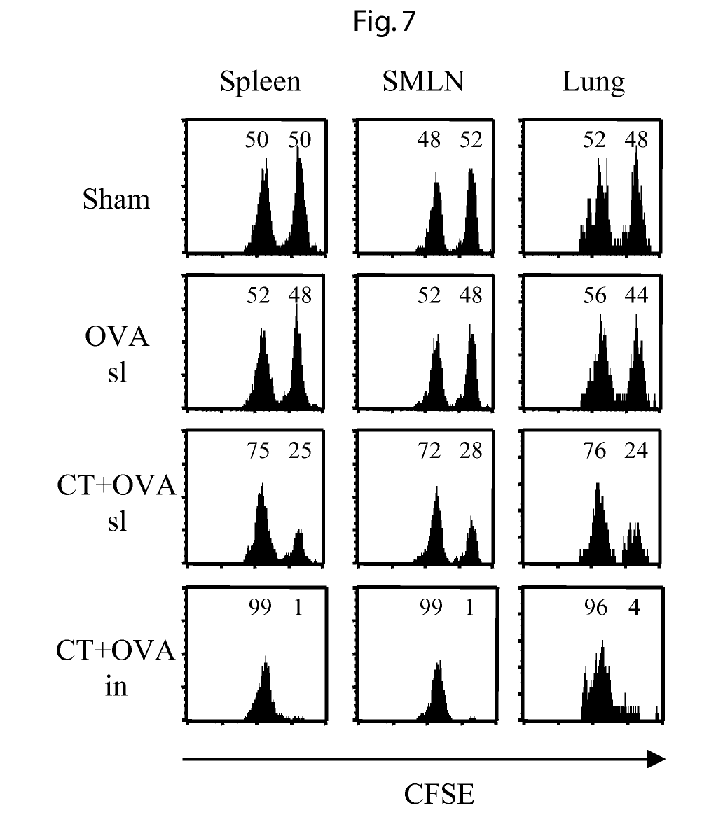

Sublingual immunization with CT and OVA also induced strong Th1 responses, as indicated by the large amounts of IFN-γ secretion after in vitro re-stimulation of SMLN and splenocytes with OVA, we evaluated whether sublingual immunization could elicit a CTL response which are known to be dependent on Th1 cells. We measured cytotoxic responses against an H2-K^{b} restricted immunodominant peptide of OVA (SIINFEKL). Naïve spleen cells pulsed with SIINFEKL peptide or unpulsed cells, and labeled with CFSEhigh or CFSElow concentration respectively, were used as target cells in an in vivo cytolysis assay. Then labeled spleen cells were infused intravenously in immunized mice 5 days after the last immunization and specific lysis was assessed 24 h or 48 h after infusion. Sublingual immunization with CT and OVA induced specific lysis in the SMLN and in the spleen (FIG. 7). Most importantly, specific lysis also occurred in the lungs after sublingual immunization with CT and OVA.

### Protective Effect of Sublingual Immunization Against Helicobacter pylori Infection

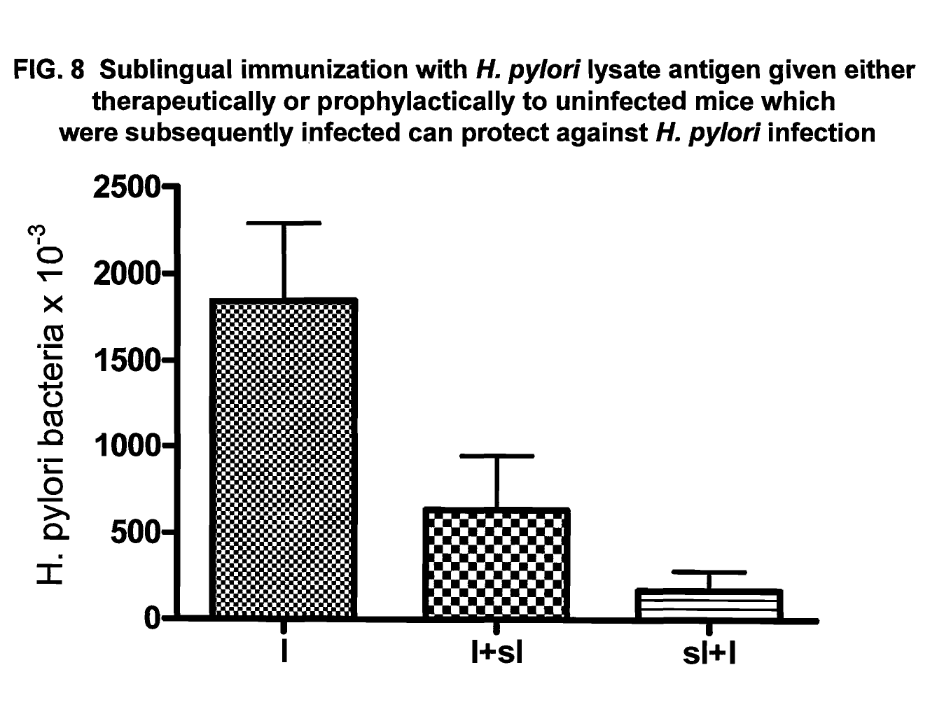

The potential of s.l. immunization, given prophylactically or therapeutically, against H. pylori infection was tested in mice. The results are shown in FIG. 8. Compared with the unimmunized infection controls (“I” in the figure) both the therapeutically immunized mice (“I+Sl”) and the prophylactically immunized animals (“Sl+I”) had a significant reduction in the magnitude of stomach infection. The prophylactically immunized animals had a greater reduction in the magnitude of infection.

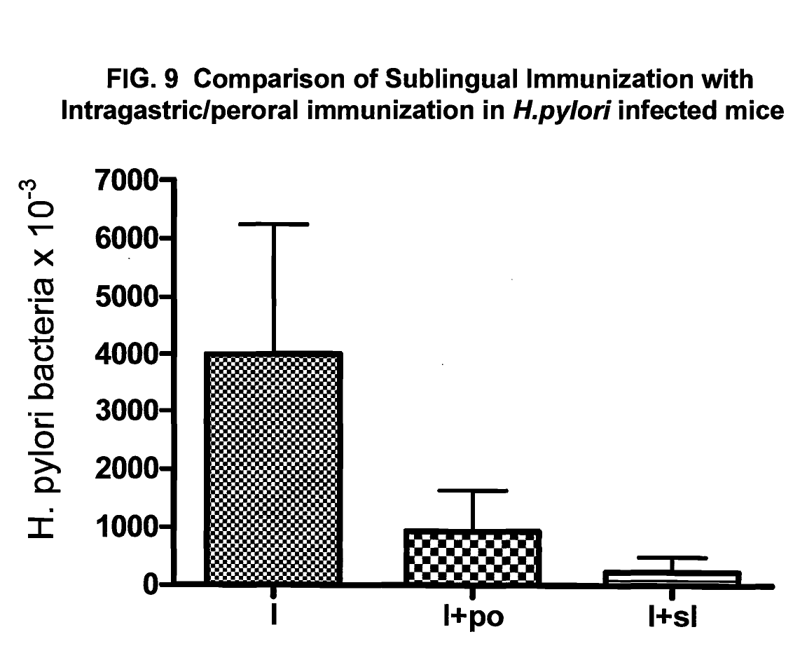

Next, the effect of s.l. immunization was compared with the effect of p.o. immunization against H. pylori infection in mice. The results are shown in FIG. 9. Compared with the infection controls (“I” in the figure), both the mice given p.o. (“I+Po”) and s.l. (“I+Sl”) immunizations showed marked reductions in the H. pylori bacterial load; the effect by the s.l. immunization showed a greater reduction in H. pylori bacterial load than that by the p.o. vaccination.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, patent and non-patent publications, product descriptions, and protocols are cited throughout this application, the disclosures of which are incorporated herein by reference in their entireties for all purposes.

What is claimed is:

1. A method for inducing both a mucosal and a systemic immune response in the respiratory, digestive or urogenital tracts to a microbial pathogen in a mammal comprising topically administering onto the sublingual mucosa an amount of an antigen effective to induce said mucosal and systemic immune responses and a pharmaceutically acceptable carrier or diluent.

2. The method of claim 1, wherein said microbial pathogen is selected from the group consisting of a virus, a bacteria, and a mycoplasma.

3. The method of claim 2, wherein said microbial pathogen is a selected from the group consisting of a live virus, a live bacteria, and a live mycoplasma.

4. The method of claim 2, wherein said microbial pathogen is selected from the group consisting of a live-attenuated virus, a live-attenuated bacteria, and a live-attenuated mycoplasma.

5. The method of claim 4, wherein said antigen is selected from the group consisting of a protein, a polysaccharide, a lipid, and a nucleic acid.

6. The method of claim 1, wherein said systemic immune response is antibody production.

7. The method of claim 6, wherein said antibody is a secretory antibody.

8. The method of claim 7, wherein said secretory antibody is selected from the group consisting of IgA, IgM and IgG.

9. The method of claim 1, wherein said systemic immune response is a cell-mediated immune response.

10. The method of claim 9, wherein said cell-mediated immune response is a cytotoxic T lymphocyte response.

11. The method of claim 9, wherein said cell-mediated immune response elicits gamma interferon production by T-lymphocytes.

12. The method of claim 1, wherein said specific antigen is co-administered with an adjuvant.

13. The method of 12, wherein said adjuvant is selected from the group consisting of cholera toxin, E. coli heat labile enterotoxin, pertussis toxin, shiga toxin, flagellin, and Toll-like receptor ligands.

14. The method of 12, wherein said adjuvant is selected from the group consisting of chemokines attracting MHC class II-expressing cells.

15. The method of claim 12, wherein said carrier contains a detergent.

16. The method of claim 12, wherein said mucosal immune response is associated with the recruitment of MHC-II-expressing antigen-presenting cells in the sublingual mucosa.

17. The method of claim 12, wherein said specific antigen is also administered with a pharmaceutically acceptable amount of a mucoadhesive or bioadhesive.

18. The method of claim 1, wherein the immune response induced is both a mucosal immune response and a systemic immune response, and wherein the immune response is induced in the respiratory tract, the digestive tract and the urogenital tract.

19. A pharmaceutical formulation or dosage form for immunizing a mammal against a microbial pathogen by topical administration onto the sublingual mucosa comprising an amount of an antigen effective for eliciting both a mucosal and a systemic immune response in the respiratory, digestive or urogenital tracts, an amount of a mucoadhesive or bioadhesive effective for prolonging the contact between the antigen and the sublingual mucosa, and a pharmaceutically acceptable carrier or diluent.

20. The pharmaceutical formulation or dosage form of claim 19, wherein said antigen is admixed with an adjuvant which combination is effective for eliciting both a mucosal and a systemic immune response against said microbial pathogen in said respiratory, digestive or urogenital tracts.

21. The pharmaceutical formulation or dosage form of claim 20, wherein said dosage form is effective to induce both a mucosal and a systemic immune response in the respiratory, digestive and urogenital tracts.
